(19) United States
(12) Patent Application Publication
(10) Pub. No.: US 2007/0004976 A1
(43) Pub. Date: Jan. 4, 2007

(54) In vivo optical measurements of hematocrit

(76) Inventors: Alex R. Zelenchuk, Stoughton, MA (US); Howard B. Kaufman, Newton, MA (US); Stephen D. Fantone, Lynnfield, MA (US)

Correspondence Address: SULLIVAN & WORCESTER LLP, ONE POST OFFICE SQUARE, BOSTON, MA, 02109, US

(21) Appl. No.: 11/293,652

(22) Filed: Dec. 2, 2005

Related U.S. Application Data
(63) Continuation-in-part of application No. 11/109,409, filed on Apr. 19, 2005.
Application No. 11/109,409 is a continuation-in-part of application No. 11/011,714, filed on Dec. 14, 2004.

(51) Int. Cl. A61B 5/00

(52) U.S. Cl. 600/322

(57) ABSTRACT

An apparatus for measuring hematocrit of a subject's blood includes an optical source that generates an optical beam that illuminates subcutaneous vessels under a subject's skin. An optical element is positioned to receive a near field portion of the optical beam that is reflected from the subcutaneous vessels under the subject's skin. A scanning mechanism positions the optical element relative to the subcutaneous vessels under the subject's skin over a plurality of relative distances. An optical detector generates a plurality of electrical signals in response to detecting the near field portion of the optical beam that is reflected from the subcutaneous vessels under the subject's skin at the plurality of relative distances between the optical element and the subcutaneous vessels. A processor determines a value of hematocrit in the subcutaneous vessels illuminated by the optical beam from the plurality of electrical signals generated by the optical detector.

### RELATED APPLICATIONS

The present application is a continuation-in-part of Ser. No. 11/109,409, filed Apr. 19, 2005 entitled “Optical Determination of In Vivo Properties”, which is a continuation-in-part of U.S. patent application Ser. No. 11/011,714, filed Dec. 14, 2004. The entire specification of U.S. patent application Ser. No. 11/011,714 and U.S. patent application Ser. No. 11/109,409 are incorporated herein by reference in their entirety.

### BACKGROUND OF THE INVENTION

The section headings used herein are for organizational purposes only and should not be construed as limiting the subject matter described in the present application.

This invention relates generally to optical measurements of in vivo properties of a solid tissue or blood. Medical personnel often need to determine properties of human or animal subject's solid tissue or blood. For example, in a diagnostic or surgical setting, it is desirable for medical personnel to know the hematocrit (Hct) of a subject's blood, which relates to the abundance of hemoglobin (Hb) and/or concentration of red blood cells in the subject's blood.

Traditional methods of determining hematocrit of a subject's blood include drawing blood from a vein in the subject's body and then centrifuging the drawn blood to separate cellular and fluid components of the blood or by mixing a chemical agent in with the blood to facilitate colorimetric measurements. Such methods are both time consuming and expensive.

### BRIEF DESCRIPTION OF THE DRAWINGS

The invention, in accordance with preferred and exemplary embodiments, together with further advantages thereof, is more particularly described in the following detailed description, taken in conjunction with the accompanying drawings. The drawings are not necessarily to scale, emphasis instead generally being placed upon illustrating principles of the invention.

FIG. 1 illustrates a schematic diagram of a tissue sample that is illuminated using the off-axis confocal method of measuring hematocrit of a subject's blood according to the present invention.

FIG. 2 illustrates an experimental apparatus that is used to simulate the method of determining hematocrit of a subject's blood according to the present invention.

FIG. 3A illustrates a schematic diagram of the optical configuration where the optical beam illuminates blood vessels that are displaced a distance from one side of the Optical Axis of the optical element.

FIG. 3B illustrates a schematic diagram of similar view of the optical configuration of FIG. 3A including the near field reflected optical beam.

FIG. 4A illustrates a schematic diagram of the optical configuration where the optical beam illuminates blood vessels that are aligned with the optical axis of the optical element.

FIG. 4B illustrates a schematic diagram of a similar view of the optical configuration of FIG. 4A including the near field reflected optical beam.

FIG. 5A illustrates a schematic diagram of the optical configuration where the optical beam illuminates blood vessels that are displaced a distance from another side of the optical axis of the optical element.

FIG. 5B illustrates a schematic diagram of a similar view of the optical configuration of FIG. 5A including the near field reflected optical beam.

FIGS. 6A and 6B illustrate experimental data for the apparatus 200 described in connection with FIG. 2, which indicates that the order of hematocrit data is “flipped” in certain ranges of z-values correlated to either near field values or far field values.

FIG. 7A-7F illustrates experimental data for the apparatus described in connection with FIG. 2, which simulates the method of determining hematocrit of a subject's blood according to the present invention.

FIG. 8 illustrates a graph of a ratio of near field to far field intensity measurements as a function of hematocrit for blood flowing in the capillary tube described in connection with FIG. 2.

FIG. 9 illustrates a schematic diagram of an apparatus for determining hematocrit of a subject's blood according to the present invention.

### DETAILED DESCRIPTION

While the present teachings are described in conjunction with various embodiments and examples, it is not intended that the present teachings be limited to such embodiments. On the contrary, the present teachings encompass various alternatives, modifications and equivalents, as will be appreciated by those of skill in the art.

For example, in some embodiments, the detailed description describes measuring hematocrit of a human's blood. It should be understood that the methods and apparatus of the present invention can be applied to measuring numerous other properties in a human or an animal subject's solid tissues or fluids. Also, the methods and apparatus of the present invention are described in connection with a single wavelength optical beam. It should be understood that the methods and apparatus of the present invention can use one or more optical beams with more than one wavelength.

It should be understood that the individual steps of the methods of the present invention may be performed in any order and/or simultaneously as long as the invention remains operable. Furthermore, it should be understood that the apparatus of the present invention can include any number or all the described embodiments as long as the invention remains operable.

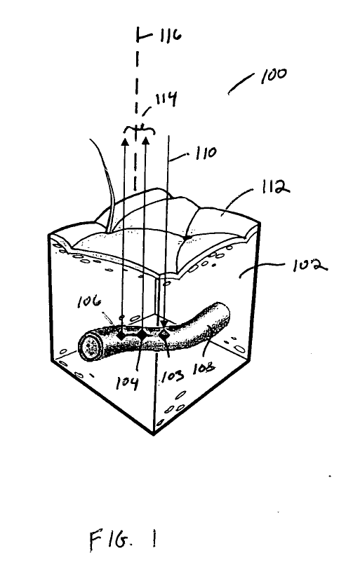

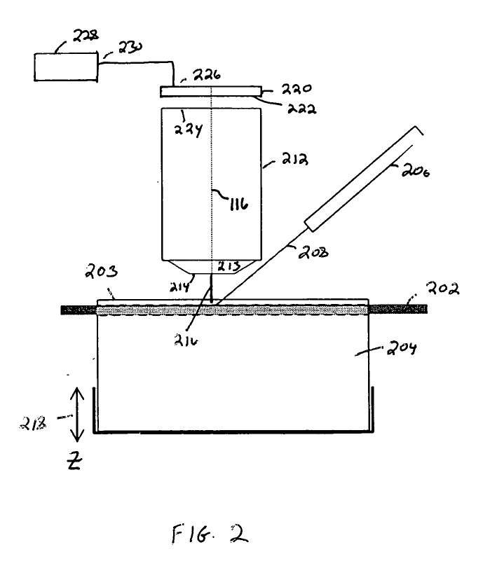

FIG. 1 illustrates a schematic diagram 100 of a tissue sample 102 that is illuminated using the off-axis confocal method of measuring hematocrit of a subject's blood according to the present invention. In this method, both the illumination volume 103 and the detection volumes 104 and 106 are created in a single subcutaneous blood vessel 108. An optical source generates an optical beam 110 that is directed to a small illumination volume of blood within the blood vessel 108 below the surface of subject's skin 112. A portion of the optical beam 110 propagates through the detection volumes 104, 106 in the blood vessel 108 and is reflected back to the surface of subject's skin 112 as shown in FIG. 1. A near field portion of the reflected optical beam 114 is reflected back to the surface of the subject's skin 112 along an optical axis 116 of an optical element, as described in connection with FIG. 2, and is detected and then analyzed to determine the hematocrit in the subject's blood.

FIG. 2 illustrates an experimental apparatus 200 that is used to simulate the method of determining hematocrit of a subject's blood according to the present invention. A blood filled glass capillary tube 202 is imbedded into a solid block 204 to simulate blood flowing through a subcutaneous blood vessel in a subject's body. A capillary tube having a 2 mm diameter was used to obtain the data presented herein. The solid block 204 is engineered to have optical properties that closely match the optical properties (μₐ and μₛ) of human skin. For example, in some experiments, the solid block 204 is formed of epoxy resin that includes tin oxide and dyes that closely match the optical properties (μₐ and μₛ) of human skin. The capillary tube 202 was embedded various distances below the surface 203 of the solid block 204, such as 0.5 mm, 0.65 mm, 0.8 mm, 0.95 mm, 1.1 mm, and 1.25 mm below the surface of the solid block 204.

An optical source 206 that generates an optical beam 208 is positioned to illuminate the blood flowing in the capillary tube 202. In some experiments, the optical source 206 was a 70 mW laser having an 808 nm wavelength. Numerous other types of optical sources can be used to perform the methods of the present invention. In some embodiments, the optical source 206 is a super luminescent diode. Experiments have shown that using a super luminescent diode results in less spurious reflections compared with using a laser and thus, results in intensity data with a relatively high signal-to-noise ratio. Such optical sources are relatively inexpensive, have long lifetimes and are extremely reliable. The optical source 206 is oriented so that the optical beam 208 strikes the blood flowing in the glass capillary tube 202 at a non-normal angle relative to the surface 210 of the capillary tube 202. An optical element 212 is positioned so that an input 214 of the optical element 212 receives a portion of the near field optical beam 216 that is reflected from blood vessels in the capillary tube 202. The term “near field” as used herein refers to the portion of the reflected optical beam that propagates along the optical axis 116 of the optical element 212. In the embodiment shown in FIG. 2, the “near field” points are approximately within 0.5 mm of the optical axis 116 of the device that detects the reflected optical beam 216.

The optical element 212 is designed to pass a desired near field portion of the optical beam 216 reflected from blood vessels in the capillary tube 202. In many embodiments, the optical element 212 includes a spatial filter that is designed to pass the desired near field portion of the reflected optical beam 216. In the embodiment shown in FIG. 2, the optical element 212 comprises a lens 213 that is positioned proximate to the capillary tube 202 so that the lens collects the desired portion of the near field optical beam that is reflected from blood vessels in the capillary tube 202.

The optical element 212 used to obtain the data presented herein was a 20× lens. The numerical aperture, which is a measure of a lens' ability to gather light and resolve fine detail, was equal to 0.42. The working distance, which is the distance from the blood vessel being illuminated to the input 214 of the optical element 212, was equal to 20 mm, and the depth of focus was equal to 1.6 μm. A relatively long working distance lens is used to reduce the angle of illumination in order to reduce forward scattering and the resulting noise.

In other embodiments, the optical element 212 is a pin hole aperture that is designed to pass the desired portion of the near field optical beam reflected from blood vessels in the capillary tube 202. In yet other embodiments, the optical element 212 includes an input to an optical fiber cable. In this embodiment, the core of the optical fiber is chosen to pass the desired portion of the near field optical beam reflected from blood vessels in the capillary tube 202. In some of these embodiments, the optical element 212 includes the lens 213 that collects the reflected optical beam 216 prior to passing the desired portion of the near field optical beam reflected from blood vessels in the capillary tube 202.

A scanning mechanism 218 is used to position the capillary tube 202 relative to the optical element 212 and the optical source 206 at a plurality of relative distances so that the optical beam 208 illuminates different illumination volumes in the capillary tube 202. The scanning mechanism 218 described in FIG. 2 translates the solid block 214 including the capillary tube 202 in the Z-direction as indicated in the figure. Experimental data is presented herein where the solid block 204 is translated in steps of 50 microns. In other embodiments, the optical element 202 and the optical source 206 are translated relative to a part of the subject's body, such as a wrist of a human subject, to obtain data for different illumination volumes in the capillary tube 202.

An optical detector 220 is used to detect the portions of the near field optical beam that are reflected from blood vessels in the capillary tube 202 and that propagate through the optical element 212. The optical detector 220 includes an optical input 222 that is coupled to the output 224 of the optical element 212. The optical detector 220 generates a plurality of electrical signals at an output 226 in response to detecting a plurality of near field portions of the optical beam 208 that are reflected from blood vessels in the capillary tube 202 as the scanning mechanism 218 changes the position the optical element 212 relative to the capillary tube 202.

A processor 228 is used to acquire the data generated by the optical detector 220. For example, the processor 228 can be a computer that includes an analog-to-digital converter or other signal processor. The processor 228 includes an input 230 that is electrically connected to the output 226 of the detector 220. The processor 228 receives the plurality of signals from the output 226 of the optical detector 220 and uses the methods described herein to determine a value of hematocrit of the blood in the vessel illuminated by the optical beam.

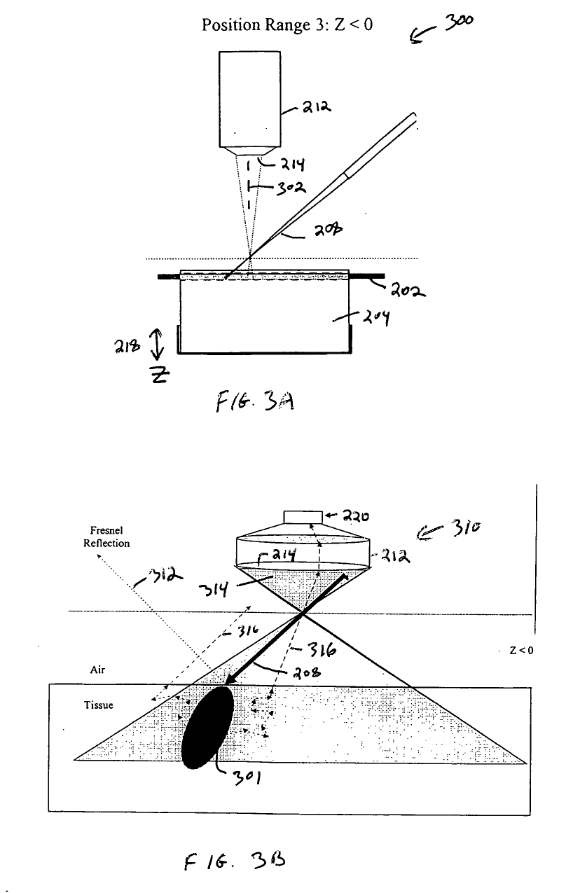

FIGS. 3-5 schematically illustrate the effect of changing the relative distance between the optical element 212 and the capillary tube 202 being illuminated by the optical beam 208 with the scanning mechanism 218. FIG. 3A illustrates a schematic diagram 300 of the optical configuration where the optical beam 208 illuminates blood vessels in the capillary tube 202 that are displaced a distance from one side of the optical axis 302 of the optical element 212 so that the near field portion of the reflected optical beam is not aligned with the input of the optical element 212.

FIG. 3B illustrates a schematic diagram 310 of a similar view of the optical configuration of FIG. 3A including the near field reflected optical beam 312. The diagram 310 indicates that the near field reflections do not intersect with the detection volume 314 of the optical element 212. The diagram 310 also indicates that the optical element 212 receives only the diffuse reflected optical beam 316. The near field reflected optical beam 312 does not propagate through the input 214 of the optical element 212. As a result, the intensity detected by the detector 220 is relatively low, originating primarily from far field illumination, and can be used to measure hematocrit only from diffuse scattering events that occur in the blood.

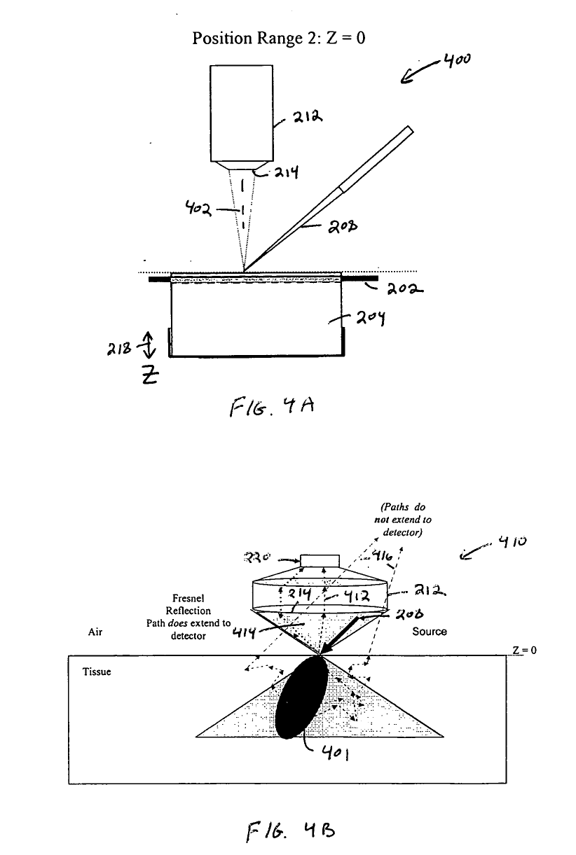

FIG. 4A illustrates a schematic diagram 400 of the optical configuration where the optical beam 208 illuminates blood vessels in the capillary tube 202 that are aligned with the optical axis 402 of the optical element 212 so that a near field portion of the reflected optical beam is aligned directly with the input 214 of the optical element 212. FIG. 4B illustrates a schematic diagram 410 of a similar view of the optical configuration of FIG. 4A including the near field reflected optical beam 412. The diagram 410 indicates that the near field reflections intersect with the detection volume 414 of the optical element 212. As a result, the intensity detected by the detector 220 is relatively high compared with the far field diffuse reflected optical beam 416. Most of the far field diffuse reflected optical beam 416 will not be detected by the detector 220 because it is not aligned with the optical axis 402. Therefore, the optical signal received by the detector 220 is generated in part by light reflected from the blood vessel 401 illuminated by the optical beam 208 and is directly related to the hematocrit in the blood vessels in the capillary tube 202 that are illuminated by the optical beam 208.

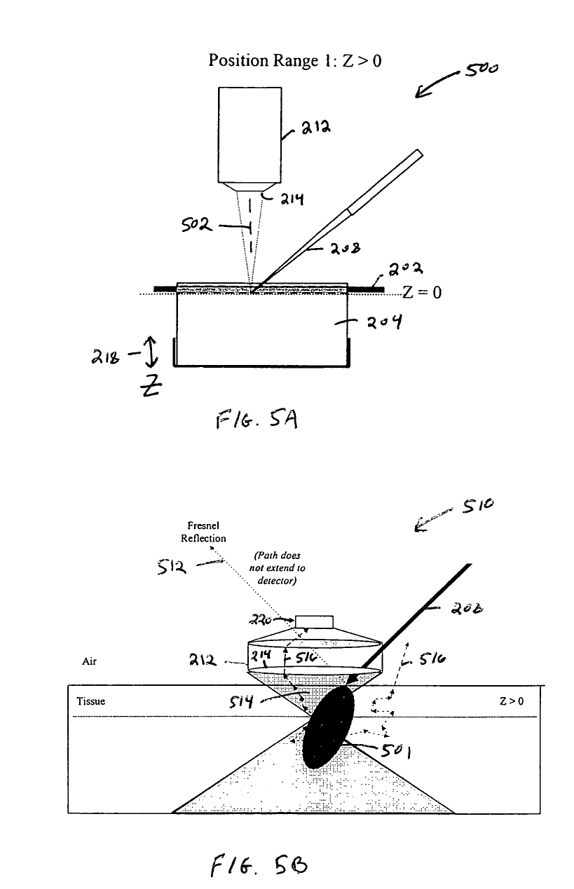

FIG. 5A illustrates a schematic diagram 500 of the optical configuration where the optical beam 208 illuminates blood vessels 501 that are displaced a distance from another side of the optical axis 502 of the optical element 212 so that the near field portion of the reflected optical beam is not aligned with the input of the optical element 212. FIG. 5B illustrates a schematic diagram 510 of a similar view of the optical configuration of FIG. 5A including the near field reflected optical beam 512. The diagram 510 indicates that the near field reflections do not intersect with the detection volume 514 of the optical element 212. The diagram 510 also indicates that the optical element 212 receives only the diffuse reflected optical beam 516. The near field reflected optical beam 512 does not propagate through the input 214 of the optical element 212. As a result, the intensity detected by the detector 220 is relatively low because it originates primarily from far field illumination and can be used to measure hematocrit only from diffuse scattering events that occur in the blood.

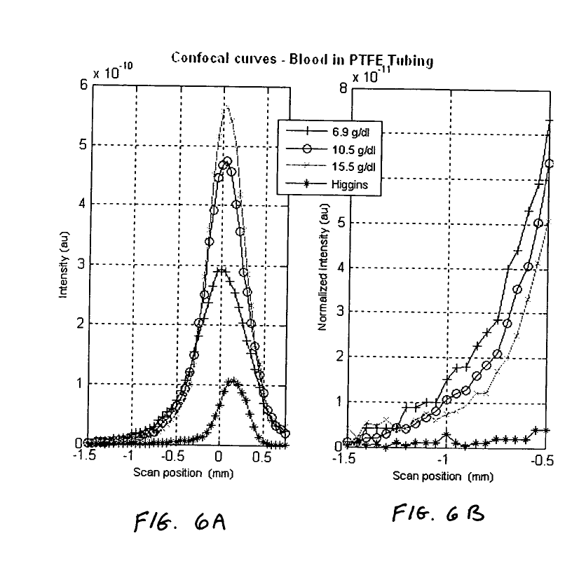

FIGS. 6A and 6B illustrate experimental data for the apparatus 200 described in connection with FIG. 2, which indicates that the order of hematocrit data is “flipped” in certain ranges of z-values correlated to either near field values or far field values. The term “flipped” as used herein refers to the observance of a change in the relationship between intensity and hematocrit from a condition where higher intensities indicate higher values of hematocrit to a condition where higher intensities indicate lower values of hematocrit.

Specifically, the data presented in FIG. 6A indicate that in the near field range where z is approximately between −0.4 mm and +0.4 mm, the highest intensity corresponds to the 15.5 g/dl specimen, followed by the next highest intensity corresponding to the 10.5 g/dl specimen, followed by the lowest intensity corresponding to the 6.9 g/dl specimen. However, the data presented in FIG. 6B indicate that in the far field range where z is less than approximately −0.5 mm (or not shown, greater than approximately +0.5 mm) the relationship between intensity and hematocrit flips such that the highest intensity corresponds to the 6.9 g/dl specimen, followed by the next highest intensity corresponding to the 10.5 g/dl specimen, followed by the lowest intensity corresponding to the 15.5 g/dl specimen. Such a far field diffuse reflectance phenomenon has been previously observed. See, for example, Schmitt J M, Mihm F G, Meindl J D. “New Methods for Whole Blood Oximetry.” Ann Biomed Eng. 1986; 14(1):35-52.

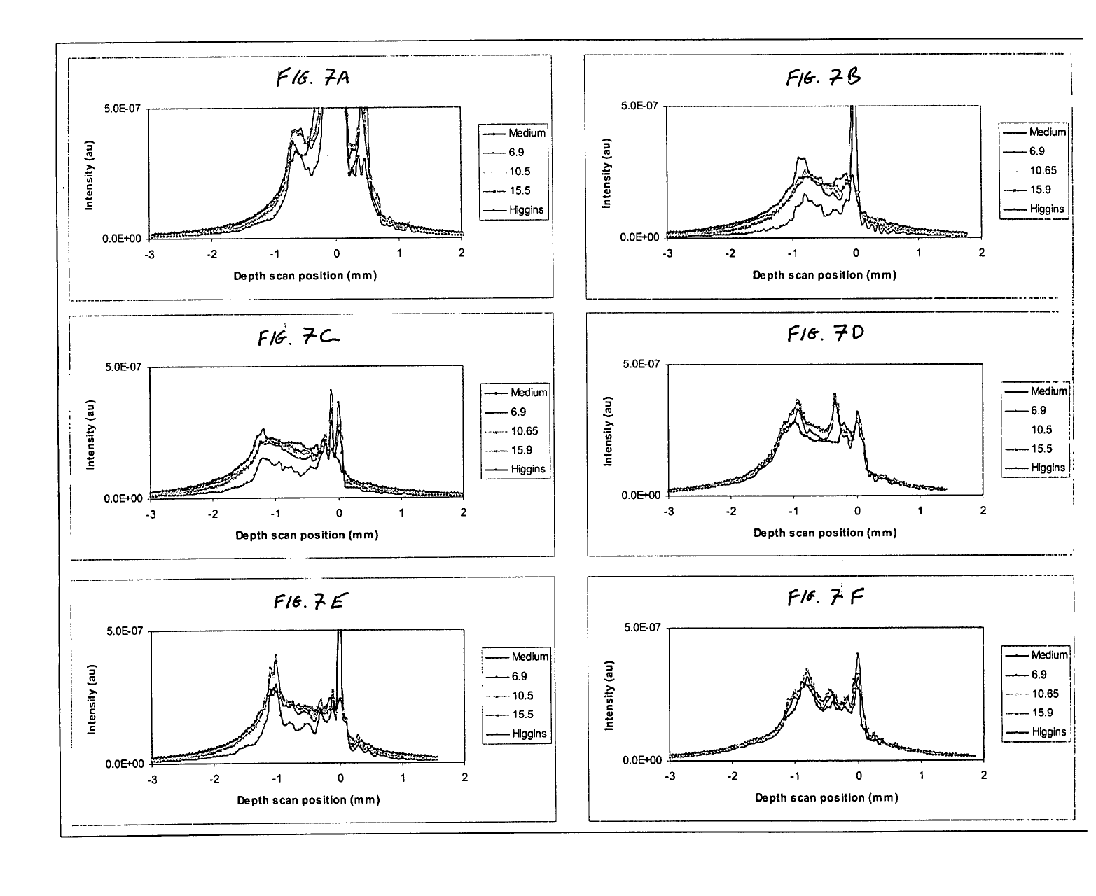

FIGS. 7A-7F illustrates experimental data for the apparatus 200 described in connection with FIG. 2, which simulates the method of determining hematocrit of a subject's blood according to the present invention. Data is presented for three different concentrations of stabilized blood samples that were passed through the capillary tube 202 in solid blocks with embedded vessels at six different depths from the surface. For comparison, data is also presented for Higgins black India ink and for the medium comprising the solid block. Data was collected by moving the solid block with the scanning mechanism 218 along the z-axis in steps of 50 microns.

FIG. 7A illustrates data for a nominal channel depth below the surface of the solid block that is equal to 0.5 mm. FIG. 7B illustrates data for a nominal channel depth below the surface of the solid block that is equal to 0.65 mm. FIG. 7C illustrates data for a nominal channel depth below the surface of the solid block that is equal to 0.8 mm. FIG. 7D illustrates data for a nominal channel depth below the surface of the solid block that is equal to 0.95 mm. FIG. 7E illustrates data for a nominal channel depth below the surface of the solid block that is equal to 1.1 mm. FIG. 7F illustrates data for a nominal channel depth below the surface of the solid block that is equal to 1.25 mm. The data presented in FIGS. 7A-7F includes a double hump that may be caused by reflections of scattered light within the optical element 212. This double hump can be eliminated by more precise alignment of detection optics as revealed by data presented in FIGS. 6A and 6B.

Many embodiments of the method of measuring hematocrit of a subject's blood according to the present invention flip the order of hematocrit data in certain ranges of relative position between the optical element 212 and the blood vessel illuminated by the optical beam 208. One explanation for why the order of hematocrit data flips in certain near field ranges as compared to far field ranges is that the type of reflections experienced by the optical beam is changing from near-surface reflections to diffuse reflections as a function of the relative z-position of the illumination and collection beams.

Near-surface reflections obey the law of Mie scattering that describes the scattering of electromagnetic radiation produced by spherical particles whose diameters are greater than 1/10 the wavelength of the scattered radiation In contrast, diffuse reflections are reflections where incoming light experiences multiple scattering events in a medium and as a result is reflected in many different directions.

Thus, one explanation for why the order of hematocrit data flips in certain ranges of the relative position is that near-surface reflections dominate in some ranges and diffuse reflections dominate in other ranges. Specifically, near-surface reflections may dominate when the optical beam 208 illuminates blood vessels that are aligned with of the optical axis 402 of the optical element 212 so that the near field portion of the reflected optical beam is aligned directly with the input 214 of the optical element 212 as described in connection with FIGS. 4A and 4B. Diffuse reflections may dominate when the optical beam 208 illuminates blood vessels that are displaced a distance from a side of the optical axis 302, 502 of the optical element 212 so that the far field portion of the reflected optical beam is aligned with the input of the optical element 212 as described in connection with FIGS. 3A, 3B, 5A and 5B.

Another possible explanation for why the order of hematocrit data flips in certain ranges of the relative position between the optical element and the blood vessel illuminated by the optical beam 208 is that there is an effective transition layer at the surface of the blood that is caused by irregular contour shaped blood vessels near the surface. This effective transition layer has a higher effective index of refraction than the deeper level of cells. The top level of cells has an effective index of refraction that is approximately equal to 1.4, which is equal to the index of refraction of a relatively high value of hematocrit and a relatively low level of blood plasma.

Near-surface reflections dominate when the optical beam 208 is reflected from the effective transition layer at the surface of the blood. The near-surface reflections from the blood vessels will increase as the concentration of hematocrit increases so that higher intensities indicate higher values of hematocrit. In contrast, deeper levels of cells have a lower effective index of refraction that is in the range of 1.33 to 1.39. Diffuse far-field reflections will dominate when the optical beam 208 is reflected from the deeper level of cells. The diffuse reflections from blood vessels will decrease as the concentration of hematocrit increases.

The separation of hematocrit measurements can be increased by computing the ratio of the intensity in the near field to the intensity in the far field. The far-field region is the region outside the near-field region, where the angular field distribution is essentially independent of distance from the source. The far field is dominated by homogeneous waves.

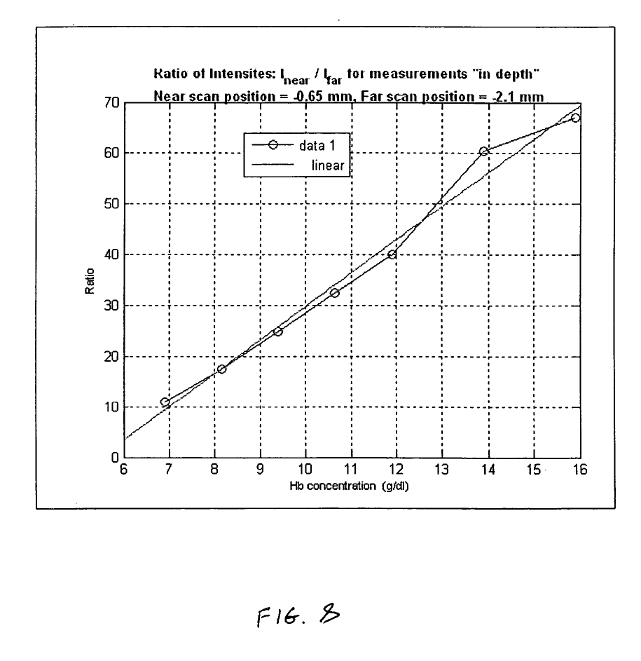

FIG. 8 illustrates a graph of a ratio of near field to far field intensity measurements as a function of hematocrit for blood flowing in the capillary tube 202 described in connection with FIG. 2. The position between the blood vessel being measured in the capillary tube 202 and the optical element 212 is 0.65 for the near field measurements. The position between the blood vessel being measured in the capillary tube and the optical element 212 is 2.1 for the far field measurements. The ratios of intensities have good separation. The graph indicates that the ratio of intensity measurements is a linear function of hematocrit values.

The data presented in FIG. 8 do not include the effect of the solid block 204 that simulates a human's skin. Similar data, however, was obtained from blood flowing in a capillary tube that is embedded into the solid block 204 described herein. Good separation of hematocrit data was also observed. The data indicates that the ratio of intensity measurements is also a linear function of hematocrit values. The slope of the data is a function of the depth of the capillary tube 202 below the surface of the solid block 204.

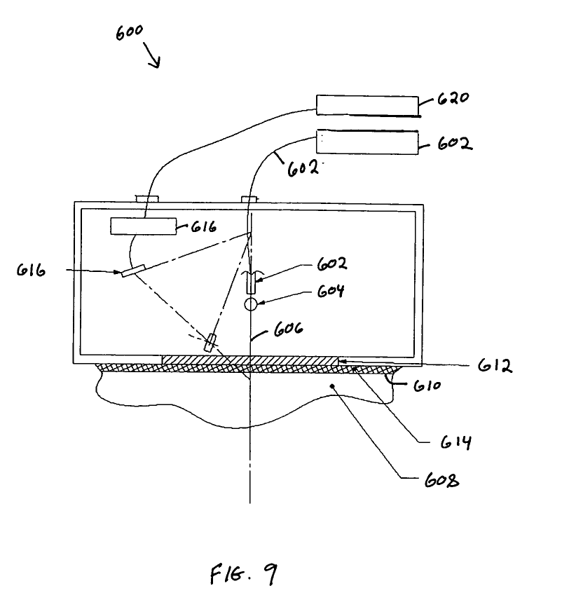

FIG. 9 illustrates a schematic diagram of an apparatus 600 for determining hematocrit of a subject's blood according to the present invention. The apparatus 600 includes a laser 602 that generates an optical beam. The output of the laser 602 is coupled to an optical fiber 602. For example, the laser 602 can be a laser diode having an optical fiber pigtail. A ball lens 604 is attached or positioned proximate to the end of the optical fiber 602. The ball lens 604 collimates the optical beam 606 generated by the laser 602 to the desired beam diameter. For example, the ball lens 604 can be a 0.6 mm ball lens that is attached to a 0.12 NA optical fiber.

The optical fiber 602 and ball lens 604 are positioned to illuminate subcutaneous vessels in the subject's tissue 608 with the optical beam 606. The apparatus 600 includes a window 612 that passes the optical beam 606 to the subcutaneous vessels in the subject's tissue 608. For example, the window 612 can be formed of quartz or sapphire. In practice, a gel 614 is used to provide an interface between the subject's skin 610 and the window 612. In some embodiments, a scanning mechanism scans the optical beam 606 relative to the subcutaneous vessels in the subject's tissue 608.

A plurality of optical elements 616 is positioned to receive a near field portion of the optical beam 606 that is reflected from the subcutaneous vessels in the subject's tissue 608. The plurality of optical elements 616 is movable as shown in FIG. 9. In the embodiment shown in FIG. 9, the plurality of optical elements 616 comprises a detector array 616. Each of the detectors in the detector array 616 has an input that is positioned at one of a plurality of unique relative distance to the subcutaneous vessels in the subject's tissue 608. Each of the detectors in the detector array 616 generates an electrical signal in response to detecting the near field intensity of the near field portion of the optical beam. A preamplifer 618 amplifies the voltage level of the signals generated by the detector array 616 to levels that are suitable for electronic processing with standard electronics.

A processor 620 is used to determine the value of hematocrit in the subcutaneous vessels illuminated by the optical beam 606 from the plurality of electrical signals generated by the detector array 616. The processor 620 has inputs that are electrically connected to the outputs of the detector array 616. The processor 620 receives the plurality of signals from the outputs of the detector array 616 and determines the hematocrit of a subject's blood from the received signals.

In other embodiments, the plurality of optical elements 616 is a bundle of fiber optic cables where each fiber optic cable is positioned so that it's input is a unique distance relative distance to the subcutaneous vessels under a subject's skin. In other embodiments, the plurality of optical elements 616 includes a plurality of pin hole apertures where each of the plurality of pin hole apertures is positioned at a unique distance relative to the subcutaneous vessels in the subject's tissue 608.

In these other embodiments, a plurality of optical detectors is used to detect the intensity of the near field portion of the optical beam 606 that is reflected from the subcutaneous vessels in the subject's tissue 608 at the unique relative distances between the optical element 616 and the subcutaneous vessels in the subject's tissue 608. Each of the plurality of optical detectors is optically coupled to an output of a respective one of the plurality of optical elements 616 and generates an electrical signal in response to detecting the near field intensity of the near field portion of the optical beam 606.

In some embodiments, the plurality of optical detectors comprises an optical multiplexer and a single optical detector. The optical multiplexer has a plurality of inputs where a respective one of the plurality of inputs is optically coupled to a respective output of the plurality of optical elements 616. The output of the optical multiplexer is optically coupled to the input of the optical detector.

The present invention includes several methods for determining the hematocrit of a subject's blood from the detected intensities of the near field portion of the optical beam that is reflected from the subcutaneous vessels under the subject's skin. In one embodiment, the detected intensities or a function of the detected intensities is compared to theoretically predicted values, such as values predicted using a photon diffusion theoretical model as described in patent application Ser. No. 11/109,409, filed Apr. 19, 2005 entitled “Optical Determination of In Vivo Properties,” which is incorporated herein by reference.

Many theoretical models for predicting hematocrit of a subject's blood from detected intensities include one or more parameters, such as the wavelength of the illuminating optical beam, the scattering and absorption cross-sections of blood vessels, and other blood components at the wavelength of the optical beam, the scattering and absorption cross-sections of subcutaneous tissue, and the distances between the light illumination volume and the light detection volume.

Theoretical models and parameters useful for such models are discussed in, e.g., Reynolds, L. O., “Optical Diffuse Reflectance and Transmittance From An Anisotropically Scattering Finite Blood Medium,” Ph.D. Thesis, Dept. Electrical Eng., Univ. of Wash., 1975; Reynolds, L. O. et al., “Diffuse Reflectance From A Finite Blood Medium: Applications To The Modeling Of Fiber Optic Catheters,” Applied Optics, 15(9), 2059-2067, 1967; and Bohren, C. F. et al., “Absorption and Scattering of Light by Small Particles,” New York, Wiley & Sons, 477-482, 1983, each of which documents is incorporated herein by reference.

Other methods of the present invention determine the hematocrit of a subject's blood from the detected intensities of the near field portion of the optical beam that is reflected from the subcutaneous vessels under the subject's skin by comparing the detected intensities to experimental values. Experimental values are obtained from one or more reference subjects having a known value for hematocrit. The known values of hematocrit can be determined using an in vitro blood analysis method. Intensity values from reference subjects having about the same hematocrit value are grouped together by averaging or by other means. Such data can be plotted in reference curves or stored in a look-up table. In these methods, intensity values are compared to the intensity values from the multiple reference subjects to determine the subject's hematocrit value or other in vivo blood property.

In some embodiments, the detected intensities are corrected for background light, such as light that exits the skin after passing only through subcutaneous tissue, which contains relatively few blood vessels. Measurements of background intensities can be obtained by adjusting the position of at least one of the optical beam 208 and the blood vessels illuminated by the optical beam 208 to a position where the optical beam does not pass through any blood vessels. This is a position where the reflected optical beam has a relatively low intensity.

The intensity measurements are corrected for background intensities by subtracting the intensity measurement of the optical beam reflected from blood vessels from the background intensity. In general, the corrected intensities are more sensitive to in vivo blood properties than uncorrected detected intensities and achieve better correlation with theoretical models. Therefore, the corrected intensities are a more accurate measure of in vivo blood property than uncorrected intensities.

In some embodiments, a ratio is calculated of measured intensities obtained at different relative positions between the optical element 212 and the subcutaneous blood vessels under the subject's skin. The different relative positions cause the optical beam 208 to illuminate blood vessels that are on and displaced various distances from the optical axis 302 of the optical element 212 so that the near field portion of the reflected optical beam is scanned relative to the input of the optical element 212. Ratios of measured intensities obtained at different relative positions are a more accurate measurement of a subject's hematocrit value or other in vivo blood property. For example, the below equation can be used to determine a subject's hematocrit value.

$$Hct = {K}_{1} \frac{I ( {λ}_{IR , {Z}_{2}} ) - {K}_{2} ( {λ}_{Green , {Z}_{2}} )}{I ( {λ}_{IR , {Z}_{1}} ) - {K}_{2} ( {λ}_{Green , {Z}_{1}} )}$$

The variables I(λ_{IR}, Z₁) and I(λ_{IR}, Z₂) are the measured near-infrared reflected intensities at a first and a second relative position Z_{1 and Z}₂ between the optical element 212 and the subcutaneous blood vessels. The variables (λ_{Green}, Z₁) and (λ_{Green}, Z₂) are the measured green light reflected intensities at the first and the second relative position between the optical element 212 and the subcutaneous blood vessels. The constants K_{1 and K}₂ are correction factors that relate the detected intensities to the actual value of the subject's hematocrit. The constants K₁ and K₂ can be determined from theoretical models, experimental data, or a combination of theoretical models and experimental data.

The intensity of green light (light having a wavelength of about 532 nm) is measured at the first and the second relative position between the optical element 212 to obtain a correction as described herein and in U.S. patent application Ser. No. 11/109,409, filed Apr. 19, 2005 entitled “Optical Determination of In Vivo Properties,” which is incorporated herein by reference. Green light does not significantly penetrate blood because blood is highly absorbing at 532 nm. Therefore, green light can be used as a reference to indicate the amount of reflections due to skin (or anything other than blood).

### Equivalents

While the present teachings are described in conjunction with various embodiments and examples, it is not intended that the present teachings be limited to such embodiments. On the contrary, the present teachings encompass various alternatives, modifications and equivalents, as will be appreciated by those of skill in the art, may be made therein without departing from the spirit and scope of the invention as defined by the appended claims.

What is claimed is:

1. An apparatus for measuring hematocrit of a subject's blood, the apparatus comprising:
a) a optical source capable of illuminating subcutaneous blood vessels;
b) an optical element capable of receiving at least a portion of an optical beam reflected from said vessels;
c) a detector for receiving input from said optical element; and
d) a processor for determining hematocrit based upon input received from said optical element.

2. The apparatus of claim 1 wherein said optical element receives a near field portion of an optical beam reflected from said vessels.

3. The apparatus of claim 1 further comprising a scanner capable of positioning the optical element at a plurality of relative distances from said vessels.

4. An apparatus for measuring hematocrit of a subject's blood, the apparatus comprising:
a) an optical source that generates an optical beam, the optical source being positioned to illuminate subcutaneous vessels under a subject's skin with the optical beam;
b) an optical element having an input that is positioned to receive a near field portion of the optical beam that is reflected from the subcutaneous vessels under the subject's skin;
c) a scanning mechanism that positions the optical element relative to the subcutaneous vessels under the subject's skin at a plurality of relative distances;
d) an optical detector having an input that is optically coupled to an output of the optical element, the optical detector generating a plurality of electrical signals at an output in response to detecting the near field portion of the optical beam that is reflected from the subcutaneous vessels under the subject's skin at the plurality of relative distances between the optical element and the subcutaneous vessels; and
e) a processor having an input that is electrically connected to the output of the detector, the processor receiving the plurality of signals generated by the optical detector and determining a value of hematocrit in the subcutaneous vessels illuminated by the optical beam from the plurality of electrical signals.

5. The apparatus of claim 4 wherein the optical source comprises a laser.

6. The apparatus of claim 4 wherein the optical source comprises a super luminescent light emitting diode.

7. The apparatus of claim 4 wherein the optical element and the optical detector comprise a single optical element that receives the near field portion of the optical beam that is reflected from the subcutaneous vessels under the subject's skin and that generates a plurality of electrical signals.

8. The apparatus of claim 4 wherein the optical element comprises a fiber optic cable.

9. The apparatus of claim 4 wherein the optical element comprises a pin hole aperture.

10. The apparatus of claim 4 further comprising a lens that is positioned proximate to an input of the optical element, the lens collecting the near field portion of the optical beam that is reflected from the subcutaneous vessels under the subject's skin.

11. The apparatus of claim 4 wherein the optical element comprises a fiber optic cable having a ball lens that is positioned proximate to an input of the fiber optical cable, the ball lens collecting the near field portion of the optical beam that is reflected from the subcutaneous vessels under the subject's skin.

12. The apparatus of claim 4 wherein the optical element comprises a plurality of optical elements, each of the plurality of optical elements being positioned at a unique relative distance to the subcutaneous vessels under the subject's skin.

13. The apparatus of claim 4 further comprising a scanning mechanism that scans the optical beam relative to the subcutaneous vessels in the subject's skin.

14. An apparatus for measuring hematocrit of a subject's blood, the apparatus comprising:
a) an optical source that generates an optical beam, the optical source being positioned to illuminate subcutaneous vessels under a subject's skin with the optical beam;
b) a plurality of optical detectors, each of the plurality of optical detectors having an input that is positioned at one of a plurality of unique relative distance to the subcutaneous vessels under a subject's skin to detect a near field portion of the optical beam that is reflected the unique relative distance, each of the plurality of optical detectors generating an electrical signal at an output in response to the detected near field portion of the optical beam; and
c) a processor having inputs that are electrically connected to the outputs of the plurality of optical detectors, the processor receiving signals from the outputs of the plurality of optical detectors and determining a value of hematocrit in the subcutaneous vessels illuminated by the optical beam from the plurality of electrical signals generated by the plurality of optical detectors.

15. The apparatus of claim 14 wherein at least some of the plurality of optical detectors comprises an optical fiber cable, an input of the optical fiber cable being positioned at the unique distance relative to the subcutaneous vessels under the subject's skin.

16. The apparatus of claim 14 wherein at least some of the plurality of optical elements comprises a pin hole aperture, an input of the pin hole aperture being positioned the unique distance relative to the subcutaneous vessels under the subject's skin.

17. The apparatus of claim 14 further comprising a scanning mechanism that scans the optical beam relative to the subcutaneous vessels in the subject's skin.

18. A method of measuring hematocrit of a subject's blood, the method comprising:
a) illuminating subcutaneous vessels under a subject's skin with an optical beam;
b) detecting a plurality of near field intensities of a portion of the optical beam that is reflected from the subcutaneous vessels under the subject's skin and generating a plurality of electrical signals in response to the detected near field intensities, each of the plurality of near field intensities being detected at a unique distance relative to the subcutaneous vessels under a subject's skin; and
c) determining a value of hematocrit of blood in the subcutaneous vessels illuminated by the optical beam from the plurality of electrical signals.

19. The method of claim 18 wherein the detecting the plurality of near field intensities comprises processing the portion of the optical beam with an optical element that passes only the near filed intensities.

20. The method of claim 18 wherein the detecting the plurality of near field intensities further comprises focusing the portion of the optical beam that is reflected from the subcutaneous vessels under the subject's skin.

21. The method of claim 18 wherein the plurality of near field intensities is detected simultaneously in time.

22. The method of claim 18 wherein the plurality of near field intensities is detected sequentially in time by changing a relative distance at which the plurality of near field intensities is detected.

23. The method of claim 18 further comprising scanning the optical beam relative to the subcutaneous vessels under a subject's skin.

24. The method of claim 18 wherein the determining the value of hematocrit of blood in the subcutaneous vessels illuminated by the optical beam from the plurality of electrical
signals comprises comparing the plurality of electrical signals to expected values obtained from experimental data.

25. The method of claim 18 wherein the determining the value of hematocrit of blood in the subcutaneous vessels illuminated by the optical beam from the plurality of electrical signals comprises comparing the plurality of electrical signals to expected values calculated from a theoretical model.

26. The method of claim 18 wherein the determining the value of hematocrit in the subcutaneous vessels illuminated by the optical beam from the plurality of electrical signals comprises determining ratios of near field intensities detected at unique distances relative to the subcutaneous vessels under a subject's skin.

27. The method of claim 18 further comprising:
a) determining a background intensity of light passing through subcutaneous tissue;
b) calculating a plurality of corrected intensities by subtracting the background intensity from the detected plurality of intensities of near field intensities; and
c) generating the plurality of electrical signals in response to the plurality of corrected intensities.

28. A method of measuring hematocrit of a subject's blood, the method comprising:
a) illuminating subcutaneous vessels under a subject's skin with an optical beam;
b) detecting a first near field intensity of a portion of an optical beam that is reflected a first distance from the subcutaneous vessels under the subject's skin and generating a first electrical signal in response to the first detected near field intensities;
c) detecting a second near field intensity of a portion of an optical beam that is reflected a second distance from the subcutaneous vessels under the subject's skin and generating a second electrical signal in response to the second detected near field intensities;
d) determining a ratio of the first and the second electrical signals; and
e) determining a value of hematocrit in the subcutaneous vessels illuminated by the optical beam from the ratio of the first and the second electrical signals.

29. The method of claim 28 further comprising:
a) determining a background intensity of light passing through subcutaneous tissue;
b) calculating a corrected first and second near field intensity by subtracting the background intensity from the detected first and second near field intensities; and
c) generating the first and the second electrical signals in response to the corrected first and second near field intensities.

30. A method of measuring hematocrit of a subject's blood, the method comprising:
a) illuminating subcutaneous vessels under a subject's skin with an optical beam;
b) detecting at least one near field intensity of a portion of an optical beam that is reflected from the subcutaneous vessels under the subject's skin and generating at least one electrical signal in response to the at least one detected near field intensity;
c) detecting at least one far field intensity of a portion of an optical beam that is reflected from the subcutaneous vessels under the subject's skin and generating at least one electrical signal in response to the at least one detected far field intensity;
d) determining at least one ratio of electrical signals generated in response to the at least one detected near field intensity and the at least one detected far field intensity; and
e) determining a value of hematocrit in the subcutaneous vessels illuminated by the optical beam from the at least one ratio.

31. The method of claim 30 further comprising:
a) determining a background intensity of light passing through subcutaneous tissue;
b) calculating a corrected at least one near field intensity by subtracting the background intensity from the detected at least one near field intensity and calculating a corrected at least one far field intensity by subtracting the background intensity from the detected at least one far field intensity; and
c) generating the at least one electrical signals in response to the corrected at least one near field intensity and the corrected at least one far field intensity.

32. An apparatus for measuring hematocrit of a subject's blood, the apparatus comprising:
a) an illuminating means for illuminating subcutaneous vessels under a subject's skin with an optical beam;
b) a detecting means for detecting a plurality of near field intensities of a portion of the optical beam that is reflected from the subcutaneous vessels under the subject's skin and generating a plurality of electrical signals in response to the detected near field intensities, each of the plurality of near field intensities being detected at a unique distance relative to the subcutaneous vessels under a subject's skin; and
c) a processing means for determining a value of hematocrit of blood in the subcutaneous vessels illuminated by the optical beam from the plurality of electrical signals.
